(19) United States
(12) Patent Application Publication
(10) Pub. No.: US 2023/0293342 A1
(43) Pub. Date: Sep. 21, 2023

(54) REUSABLE ICE PACK FOR A CAST

(71) Applicant: Joseph Hamad, Harrisburg, PA (US)

(72) Inventor: Joseph Hamad, Harrisburg, PA (US)

(21) Appl. No.: 18/124,441

(22) Filed: Mar. 21, 2023

Related U.S. Application Data
(60) Provisional application No. 63/321,922, filed on Mar. 21, 2022.

(51) Int. Cl. A61F 7/10

(52) U.S. Cl. CPC A61F 7/10; A61F 2007/0209

(57) ABSTRACT

A reusable cooling pack for a cast that includes a body with a sidewall of a substantially rigid material, of a slender configuration, with an upper end, with a lower end having a cap selectively removably coupled to the lower end, a body length separating the upper and lower ends, defining an internal channel enclosed by the sidewall and spanning longitudinally from the lower end of the body and along the body length, and with a handle member coupled to the body and extending outwardly from the upper end, wherein the internal channel encapsulated the body and the cap and configured to receive a temperature-modulating solution therein for cooling the user when inserted inside a user's cast.

### FIELD OF THE INVENTION

The present invention relates generally to therapeutic and/or medical accessories, and more particularly, to a reusable cooling pack operably configured to be inserted between inner wall of cast and affected body part.

### BACKGROUND OF THE INVENTION

Users in a cast are often told to use ice or other cooling packs to reduce pain, inflammation, cramping, itching, and swelling. However, the physical and chemical composition of existing reusable ice packs often renders it difficult, if not altogether impossible, to insert and fit the ice pack within a cast, i.e., in the limited free space between the wall of the cast and the body part. For this reason, existing prior art is not structurally configured to address this issue. Applying an ice pack to the outside of a cast, meanwhile, does not effectively or efficiently provide cooling or pain relief to the user, as a cast is generally too thick for any significant relief to be felt by the user. Casts are partly made from fiberglass or plaster, which form the hard layer that protects the injured limb and keeps it immobilized. While this material composition protects the injured limb, it also serves as a thick physical barrier that prevents any cooling or pain relief from being felt on the confined body part. Consequently, users are often forced to turn to other forms of pain treatment and discomfort relief such as pain medications, which may give rise to extensive and serious consequences, e.g., addiction(s). Most known cooling packs are too flexible or geometrically impossible to insert within a user's cast in any efficient or effective manner.

Therefore, a need exists to overcome the problems with the prior art as discussed above.

### SUMMARY OF THE INVENTION

The invention provides a reusable cooling pack for a cast that overcomes the hereinafore-mentioned disadvantages of the heretofore-known devices and methods of this general type and that is configured to be used on or around body parts that are confined within a cast. The present invention is operably configured to fit inside the limited free space between the wall of a user's cast and the body part that is confined within the cast. In this way, cooling pain relief can be more directly administered and applied to the subject area on the body, reducing the need for alternate forms of pain management such as prescription medications. Embodiments of the present invention also allow the portable assembly to be reused numerous times and easily stored and transported.

With the foregoing and other objects in view, there is provided, in accordance with the invention, a reusable cooling pack for a cast having a body with a sidewall of a substantially rigid material, of a slender configuration, with an upper end, with a lower end opposite the upper end and with a cap selectively removably coupled to the lower end, a body length separating the upper and lower ends, defining an internal channel enclosed by the sidewall and spanning longitudinally from the lower end of the body and along the body length, and with a handle member coupled to the body and extending outwardly from the upper end, wherein the internal channel is encapsulated the body and the cap and is configured to receive a temperature-modulating solution therein for cooling a user's limb while the body is inserted inside the cast.

In accordance with a further feature of the present invention, the temperature-modulating solution is formed with at least one of water, propylene glycol, and sodium carboxymethyl cellulose.

In accordance with another feature, an embodiment of the present invention includes the temperature-modulating solution consists of at least 20% propylene glycol.

In accordance with a further feature, an embodiment of the present invention includes a cloth pouch sized and shaped to receive at least 75% of the body length.

In accordance with yet another feature of the present invention, the cap is selectively removably coupled to the lower end in a watertight configuration.

In accordance with an exemplary feature of the present invention, the body is of a unitary configuration and the sidewall is of a monolithic structural configuration.

In accordance with a further feature of the present invention, the body is formed in a flattened cylindrical configuration.

In accordance with yet another feature of the present invention, the body is formed in an elongated accurate configuration.

In accordance with another feature of the present invention, the handle member is of a substantially rigid material and is coupled to the body in a fixed configuration. The handle member may also form a loop for grasping by the user. Additionally, the handle member is fixed to the body, centrally disposed, and longitudinally oriented relative to the body.

In accordance with a further feature of the present invention, the internal channel includes the temperature-modulating solution disposed therein.

In accordance with yet another feature of the present invention, the internal channel spans greater than 75% of the body length.

In accordance with a further feature of the present invention, the body has a max body length of 18 cm, a max width of 10 cm, and a max depth of 2 cm.

Also in accordance with the present invention, a reusable cooling pack for a cast is disclosed that includes a body with a sidewall of a monolithic structural configuration and of a substantially rigid material, of a slender configuration, with an upper end, with a lower end opposite the upper end and with a cap selectively removably coupled to the lower end, a body length separating the upper and lower ends, defining an internal channel enclosed by the sidewall and spanning longitudinally from the lower end of the body and along the body length, and with a handle member fixedly coupled to the body in a longitudinal orientation and extending outwardly from the upper end, wherein the internal channel is encapsulated the body and the cap and houses a temperature-modulating solution therein for cooling a user's limb.

Although the invention is illustrated and described herein as embodied in a reusable cooling pack or structure for a cast, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time. Also, for purposes of description herein, the terms “upper”, “lower”, “left,” “rear,” “right,” “front,” “vertical,” “horizontal,” and derivatives thereof relate to the invention as oriented in the figures and is not to be construed as limiting any feature to be a particular orientation, as said orientation may be changed based on the user's perspective. Furthermore, unless otherwise specified, there is no intention to be bound by any expressed or implied theory presented in the preceding technical field, background, brief summary or the following detailed description.

As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. In this document, the term “longitudinal” should be understood to mean in a direction corresponding to an elongated direction of the assembly or to-and-from the opposing ends of the assembly.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1 is a perspective view of a reusable cooling pack for a cast in accordance with an exemplary embodiment of the present invention;

FIGS. 2-3 are front and rear elevational views, respectively, of the reusable cooling pack depicted in FIG. 1;

FIG. 4 is a side elevational view of the reusable cooling pack depicted in FIG. 1;

FIG. 5 is a top plan view of the reusable cooling pack depicted in FIG. 1;

FIG. 6 is a bottom plan view of the reusable cooling pack depicted in FIG. 1;

FIG. 7 is a partially cross-sectional view of the reusable cooling pack depicted in FIG. 1 and selectively removable cloth cover in accordance with one embodiment of the present invention;

FIG. 8 is a partially cross-sectional view of the reusable cooling pack depicted in FIG. 1 with the cap removed therefrom; and

FIG. 9 is a bottom plan view of a reusable cooling pack in accordance with another embodiment of the present invention.

### DETAILED DESCRIPTION OF THE INVENTION

While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale. It is to be understood that the disclosed embodiments are merely exemplary of the invention, which can be embodied in various forms.

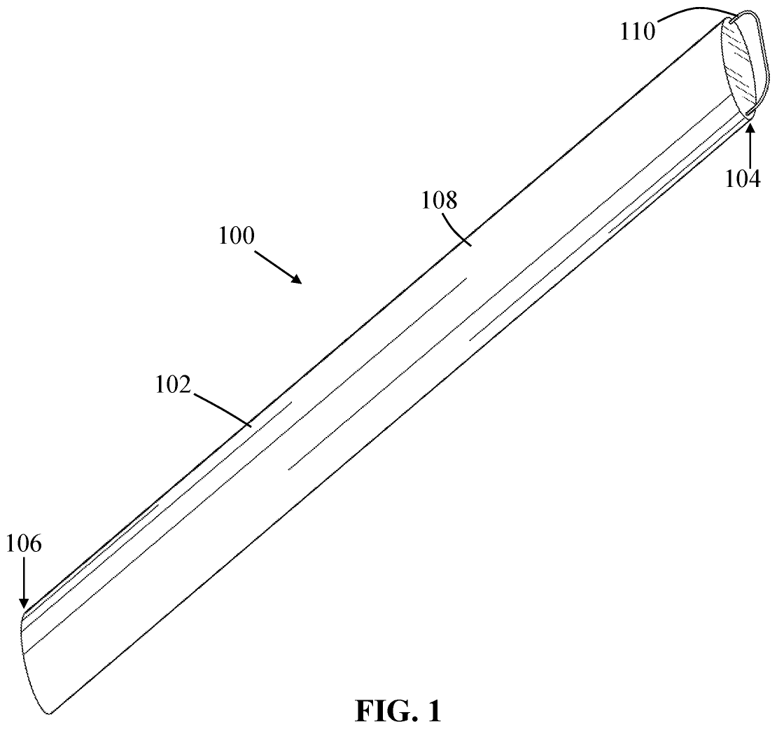

Referring now to FIG. 1, one embodiment of the present invention is shown in a perspective view. FIG. 1 shows several advantageous features of the present invention, but, as will be described below, the invention can be provided in several shapes, sizes, combinations of features and components, and varying numbers and functions of the components. The present invention provides a reusable ice pack assembly for a cast 100 (referred to as “assembly 100” hereinafter for brevity) that substantially improves a user's level of physical comfort when wearing the cast. Specifically, the assembly 100 is directly applied to, or placed on, the confined body part inside the cast to more effectively, quickly, and easily reduce pain, inflammation, cramping, itching, and swelling. In turn, this beneficially reduces the need for dependency on pain medication as well as the potential for addiction.

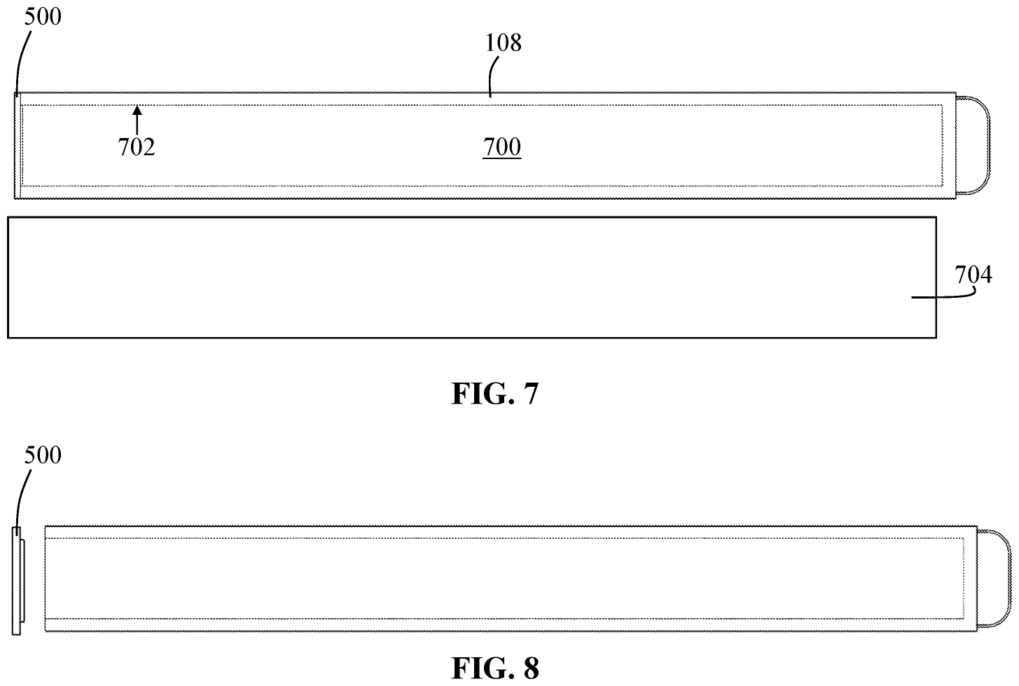

The assembly 100 includes a body 102 with an upper end 104, a lower end 106 opposite the upper end 104, a substantially rigid sidewall 108, an internal channel 700 defined by the body 102, namely the substantially rigid outer wall 108, a selectively removable cap 500 operably configured to seal the internal channel 700, and a handle member 110 extending outwardly from the upper end 104 of the body 102. The handle member 110 is preferably coupled to the body 102 in a fixed configuration, i.e., not capable of rotation or lateral movement, and is also preferably centrally located to ensure equal force is applied to the body 102 through the handle 110. Said another way, the handle member 110 is affixed to the body 102 such that it is longitudinally oriented relative to the body 102 and disposed such that it is at least partially disposed over the geometric center of the body 102. The structural rigidity of the body 102 permits the user to insert and remove the assembly 100 from a user's cast effectively and efficiently. Beneficially, the internal channel 700 defined by the body 102 is shaped and sized to receive and house a temperature-modulating solution disposed therein, e.g., frozen water, gel, etc. As best seen in FIGS. 7-8, the internal channel 700 can be seen defined by the sidewall 108 and spanning from the lower end 106 to a point proximal to the upper end 104. “Proximal” is defined as at or near (within 10% of the overall longitudinal length) of a referring structure. Further, the internal channel 700 preferably spans greater than 75% of the body length to provide greater surface area to cool the user.

The cap 500 may be selectively removably coupled to the lower end 106 in a watertight configuration using, for example, an elastomeric seal surrounding a portion of the cap 500 or surrounding an inner surface 702 of the wall 108 defining the internal channel 700. In one beneficial embodiment, the cap 500 is coupled to the body 102 in a snap-fit configuration, wherein the cap 500 includes a flange surrounding a perimeter of the cap 500 and the inner surface 700 includes a notch surrounding a perimeter thereon. In said embodiment, the user may be required to assert a tensile or longitudinal force of approximately 21 bf to attach and detach the cap 500 from the body 102.

The substantially rigid body 102, namely the sidewall 108, is substantially hard, rigid, stiff, or inflexible to beneficially improve the maneuverability of the body 102 within the cast, i.e., to allow the body 102 to slide between the inner wall of the cast and the confined body part within the cast without significant resistance and flexing of the body 102. Accordingly, the substantially rigid sidewall 108 of the body 102 may be of a polypropylene or high-density polyethylene (HDPE) composition in a preferred embodiment of the invention. Due to the confined, limited, and tight spacing that is generally created between the cast and the confined body part, the body 102 is preferably no larger in size or having max dimensions than approximately 4 in (10 cm)×1.7 in (2 cm)×6.8 in (18 cm) (width 502×depth 504×length 200) to effectuate the purpose of the present invention. Said another way, the body 102 may be of a slender configuration, wherein the length 200 is individually greater than both the width 502 and the depth 504 of the body 102. The length 200 can be seen separating the upper and lower ends 104, 106 of the body 102. The sidewall 108 is also preferably of a thermally conductive material, i.e., generally considered by those of skill in the art as a non-insulating material, thereby providing a cooling effect to the user. The sidewall 108 also preferably has a unitary thickness spanning along the body length 200 of approximately 0.5-1.5 in. The rigidity or stiffness of the sidewall 108, e.g., measured through the Young's Modulus, preferably ranges from 0.5-1.5 GPa. The Young's Modulus may also be no less than 0.1 GPa and have a range of approximately 0.5-8 GPa.

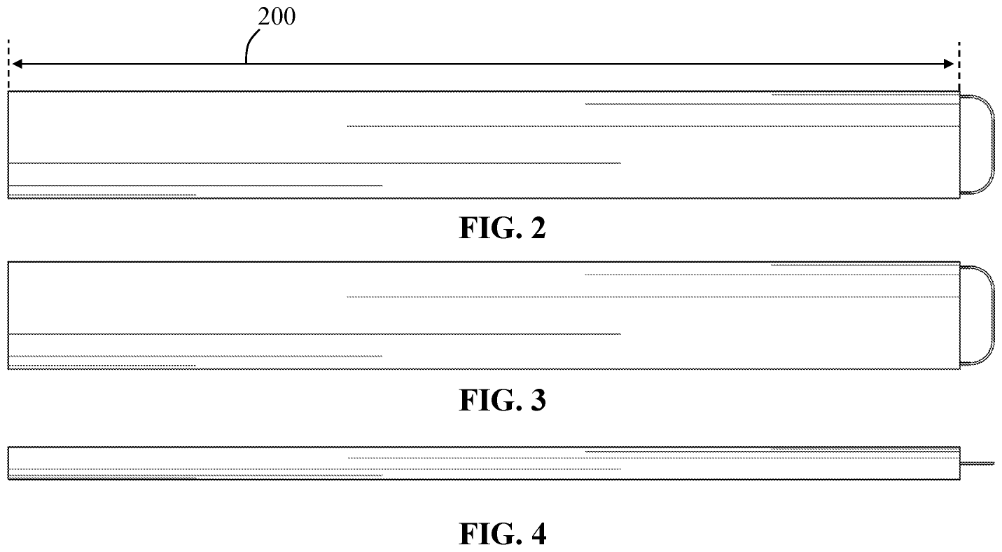

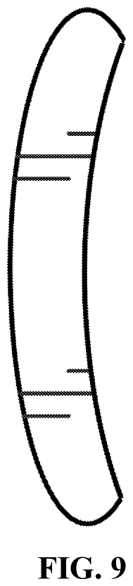

In preferred embodiments, the body 102 is either formed of a flattened cylindrical configuration (exemplified in FIGS. 1-6) or an elongated arcuate configuration (wherein FIG. 9 depicts an arcuate shape and FIG. 1 depicts an elongation of the body 102). Said differently, the body 102 may be formed as an oblong oval configuration, wherein said above-described shapes and configuration advantageously and functionally enable the user to easily and effectively insert and remove the body 102 to and from, respectively, the user's cast. The specific shape, size, and dimensions of the body 102 may vary in alternate embodiments, however, to best suit or accommodate the particular body part and area that it is intended to be used with, e.g., the leg, arm, elbow, ribs, etc.

The body 102 is beneficially unitary, i.e., operably configured to be moved as a singular piece of material. In one embodiment, the body 102, namely the sidewall 104, is formed in a monolithic structural configuration, i.e., as a single piece of material using, for example, injection molding or casting, thereby reducing the likelihood of failure after repeated use.

Disposed on the upper end 104 of the body 102 is the handle member 110 which beneficially improves the ease with which the body 102 may be slid into and positioned within the free space between the cast and the confined body part. A user may use the handle member 110 to better maneuver and position the body 102 within the cast. In preferred embodiments, the handle member 110 is also of a substantially rigid or hard material, e.g., a plastic material composition such as polypropylene, HDPE, etc. The handle member 110 can also be seen forming a loop shaped and sized for grasping by the user and inserting and removing the body 102 relative to the user's cast.

The substantially rigid outer wall 108 defines the internal channel 700 within the body 102, wherein the internal channel 700 is sized and shaped to retain the temperature-modulating solution therein. The temperature-modulating solution is operably configured to maintain a cool temperature during use (e.g., between 20° F. and 80° F.) and may consist of a variety of different active and inactive ingredients. In accordance with one exemplary embodiment, the temperature-modulating solution consists of 60% to 70% water, 20% to 35% propylene glycol, and <5% sodium carboxymethyl cellulose. Propylene glycol is the main ingredient used to lower the temperature of the body 102. Additional potential ingredients include water, an alternative ingredient to lower the freezing temperature, a thickening agent, silica gel, and non-toxic blue coloring. In an alternate embodiment, the temperature-modulating solution may consist of small gel beads made of sodium polyacrylate or eutectic gel.

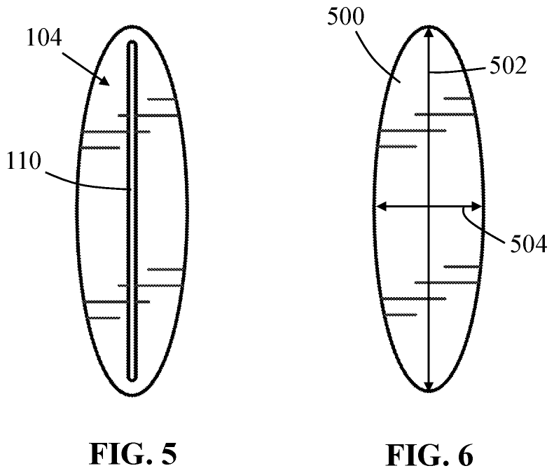

As best depicted in FIG. 5, the selectively removable cap 500 disposed on the lower end 106 of the body 102 is designed to seal and unseal the internal channel 700 if needed, e.g., to replace, remove, add to, or empty the temperature-modulating solution. In accordance with an exemplary embodiment, the assembly 100 further comprises a cloth pouch 702 sized and shaped to receive the body 102 of the assembly 100. The cloth pouch is designed to prevent skin damage, irritation, and/or discomfort caused by the direct physical contact between the body 102 of the assembly 100 and the skin of the confined area of the user's body during use. Said differently, the cloth pouch 702 is shaped and sized to receive at least 75% of the body length 200.

To utilize the assembly 100, the assembly 100 (with the exception of the cloth pouch) must be placed in the freezer until thoroughly frozen (approximately 1-4 hours). Once thoroughly frozen, the cloth pouch is positioned over the body 102 before being inserted between the cast and the confined or affected area of the body. Relief is felt almost instantly. Beneficially, the assembly 100 may reduce dependency on pain medication as well as the potential for addiction. Once cold therapy is complete, the body 102 is pulled out using the handle member 110 and placed back in the freezer for future use. A variety of shapes and sizes are possible for use on different areas of the body.

What is claimed is:

1. A reusable cooling pack for a cast comprising:
a body with a sidewall of a substantially rigid material, of a slender configuration, with an upper end, with a lower end opposite the upper end and with a cap selectively removably coupled to the lower end, a body length separating the upper and lower ends, defining an internal channel enclosed by the sidewall and spanning longitudinally from the lower end of the body and along the body length, and with a handle member coupled to the body and extending outwardly from the upper end, the internal channel encapsulated the body and the cap and configured to receive a temperature-modulating solution therein.

2. The reusable cooling pack for a cast according to claim 1, wherein the temperature-modulating solution comprises water, propylene glycol, and sodium carboxymethyl cellulose.

3. The reusable cooling pack for a cast according to claim 1, wherein the temperature-modulating solution consists of at least 20% propylene glycol.

4. The reusable cooling pack for a cast according to claim 1, further comprising a cloth pouch sized and shaped to receive at least 75% of the body length.

5. The reusable cooling pack for a cast according to claim 1, wherein the cap is selectively removably coupled to the lower end in a watertight configuration.

6. The reusable cooling pack for a cast according to claim 1, wherein the body is of a unitary configuration and the sidewall is of a monolithic structural configuration.

7. The reusable cooling pack for a cast according to claim 1, wherein the body is formed in a flattened cylindrical configuration.

8. The reusable cooling pack for a cast according to claim 1, wherein the body is formed in an elongated accurate configuration.

9. The reusable cooling pack for a cast according to claim 1, wherein the handle member is of a substantially rigid material and is coupled to the body in a fixed configuration.

10. The reusable cooling pack for a cast according to claim 9, wherein the handle member forms a loop for grasping by the user.

11. The reusable cooling pack for a cast according to claim 10, wherein the handle member is fixed to the body, centrally disposed, and longitudinally oriented relative to the body.

11. The reusable cooling pack for a cast according to claim 1, wherein the internal channel includes the temperature-modulating solution disposed therein.

12. The reusable cooling pack for a cast according to claim 1, wherein the internal channel spans greater than 75% of the body length.

13. The reusable cooling pack for a cast according to claim 1, wherein the body has a max body length of 18 cm, a max width of 10 cm, and a max depth of 2 cm.

14. A reusable cooling pack for a cast comprising:
a body with a sidewall of a monolithic structural configuration and of a substantially rigid material, of a slender configuration, with an upper end, with a lower end opposite the upper end and with a cap selectively removably coupled to the lower end, a body length separating the upper and lower ends, defining an internal channel enclosed by the sidewall and spanning longitudinally from the lower end of the body and along the body length, and with a handle member fixedly coupled to the body in a longitudinal orientation and extending outwardly from the upper end, the internal channel encapsulated the body and the cap and housing a temperature-modulating solution therein.

15. The reusable cooling pack for a cast according to claim 14, wherein the internal channel spans greater than 75% of the body length.

16. The reusable cooling pack for a cast according to claim 14, wherein the body has a max body length of 18 cm, a max width of 10 cm, and a max depth of 2 cm.

17. The reusable cooling pack for a cast according to claim 14, wherein the cap is selectively removably coupled to the lower end in a watertight configuration.

18. The reusable cooling pack for a cast according to claim 14, wherein the body is formed in a flattened cylindrical configuration.

19. The reusable cooling pack for a cast according to claim 14, wherein the body is formed in an elongated accurate configuration.
